(19) United States
(12) Patent Application Publication
(10) Pub. No.: US 2006/0147177 A1
(43) Pub. Date: Jul. 6, 2006

(54) Fluoropolymer coating compositions with olefinic silanes for anti-reflective polymer films

(76) Inventors: Naiyong Jing, Woodbury, MN (US); Chuntao Cao, Woodbury, MN (US); Tatsuo Fukushi, Woodbury, MN (US); Timothy J. Tatge, Minneapolis, MN (US); William D. Coggio, Hudson, WI (US); Christopher B. Walker JR., St. Paul, MN (US); Thomas P. Klun, Lakeland, MN (US); William J. Schultz, North Oaks, MN (US); Zai-Ming Qiu, Woodbury, MN (US)

Correspondence Address: 3M INNOVATIVE PROPERTIES COMPANY, PO BOX 33427, ST. PAUL, MN, 55133-3427, US

(21) Appl. No.: 11/026,752

(22) Filed: Dec. 30, 2004

(51) Int. Cl. B32B 27/00; B32B 27/20; B32B 27/30; B05D 5/06; C07C 61/08

(52) U.S. Cl. 385/147; 428/421; 428/323; 428/447; 428/422; 428/522; 427/162; 562/400

(57) ABSTRACT

An economic, optically transmissive, stain and ink repellent, durable low refractive index fluoropolymer composition for use in an antireflection film or coupled to an optical display. In one aspect of the invention, the composition is formed from the reaction product of a fluoropolymer, a C═C double bond group containing silane ester agent, and an optional multi-olefinic crosslinker. In another aspect of the invention, the composition further includes surface modified inorganic nanoparticles. In another aspect, the multi-olefinic crosslinker is an alkoxysilyl-containing multi-olefinic crosslinker.

### TECHNICAL FIELD AND INDUSTRIAL APPLICABILITY OF THE INVENTION

The present invention relates to antireflective films and more specifically to low refractive index fluoropolymer coating compositions for use in antireflection polymer films.

### BACKGROUND OF THE INVENTION

Antireflective polymer films (“AR films”) are becoming increasingly important in the display industry. New applications are being developed for low reflective films applied to substrates of articles used in the computer, television, appliance, mobile phone, aerospace and automotive industries.

AR films are typically constructed by alternating high and low refractive index (“RI”) polymer layers in order to minimize the amount of light that is reflected from the optical display surface. Desirable product features in AR films for use on optical goods are a low percentage of reflected light (e.g. 1.5% or lower) and durability to scratches and abrasions. These features are obtained in AR constructions by maximizing the delta RI between the polymer layers while maintaining strong adhesion between the polymer layers.

It is known that the low refractive index polymer layers used in AR films can be derived from fluorine containing polymers (“fluoropolymers” or “fluorinated polymers”). Fluoropolymers provide advantages over conventional hydrocarbon-based materials relative to high chemical inertness (in terms of acid and base resistance), dirt and stain resistance (due to low surface energy) low moisture absorption, and resistance to weather and solar conditions.

The refractive index of fluorinated polymer coating layers can be dependent upon the volume percentage of fluorine contained within the layer. Increased fluorine content in the layers typically decreases the refractive index of the coating layer. However, increasing the fluorine content of fluoropolymer coating layers can decrease the surface energy of the coating layers, which in turn can reduce the interfacial adhesion of the fluoropolymer layer to other polymer or substrate layers to which the layer is coupled.

Thus, it is highly desirable to form a low refractive index layer for an antireflection film having increased fluorine content, and hence lower refractive index, while improving interfacial adhesion to accompanying layers or substrates.

### SUMMARY OF THE INVENTION

The present invention provides an economic and durable low refractive index fluoropolymer composition for use as a low refractive index film layer in an antireflective film for an optical display. The low refractive index composition forms layers having strong interfacial adhesion to a high index refractive layer and/or a substrate material.

In one aspect of the invention, a low refractive index layer is formed from the reaction product of a reactive fluoropolymer, a C═C double bond containing silane agent such as a multi-acrylate, 3-(trimethoxysilyl)propyl methacrylate and/or vinyltrimethoxysilane, and an optional multi-olefinic crosslinker.

The term “reactive fluoropolymer”, or “functional fluoropolymer” will be understood to include fluoropolymers, copolymers (e.g. polymers using two or more different monomers), oligomers and combinations thereof, which contain a reactive functionality such as a halogen containing cure site monomer and/or a sufficient level of unsaturation. This functionality allows for further reactivity between the other components of the coating mixture to facilitate network formation during cure and improve further the durability of the cured coating.

Further, the mechanical strength and scratch resistance the low refractive index composition can be enhanced by the addition of surface functionalized nanoparticles into the fluoropolymer compositions. Providing functionality to the nanoparticles enhances the interactions between the fluoropolymers and such functionalized particles.

The present invention also provides an article having an optical display that is formed by introducing the antireflection film having a layer of the above low refractive index compositions to an optical substrate. The resultant optical device has an outer coating layer that is easy to clean, durable, and has low surface energy.

Other objects and advantages of the present invention will become apparent upon considering the following detailed description and appended claims, and upon reference to the accompanying drawings.

### BRIEF DESCRIPTION OF THE DRAWINGS

FIG. 1 is perspective view of an article having an optical display; and

FIG. 2 is a sectional view of the article of FIG. 1 taken along line 2-2 illustrating an antireflection film having a low refractive index layer formed in accordance with a preferred embodiment of the present invention.

### DETAILED DESCRIPTION AND PREFERRED EMBODIMENTS OF THE INVENTION

For the following defined terms, these definitions shall be applied, unless a different definition is given in the claims or elsewhere in the specification.

The term “polymer” will be understood to include polymers, copolymers (e.g. polymers using two or more different monomers), oligomers and combinations thereof, as well as polymers, oligomers, or copolymers that can be formed in a miscible blend.

As used herein, the term “ceramer” is a composition having inorganic oxide particles, e.g. silica, of nanometer dimensions dispersed in a binder matrix. The phrase “ceramer composition” is meant to indicate a ceramer formulation in accordance with the present invention that has not been at least partially cured with radiation energy, and thus is a flowing, coatable liquid. The phrase “ceramer composite” or “coating layer” is meant to indicate a ceramer formulation in accordance with the present invention that has been at least partially cured with radiation energy, so that it is a substantially non-flowing solid. Additionally, the phrase “free-radically polymerizable” refers to the ability of monomers, oligomers, polymers or the like to participate in crosslinking reactions upon exposure to a suitable source of curing energy.

The term “low refractive index”, for the purposes of the present invention, shall mean a material when applied as a layer to a substrate forms a coating layer having a refractive index of less than about 1.5, and more preferably less than about 1.45, and most preferably less than about 1.42.

The term “high refractive index”, for the purposes of the present invention, shall mean a material when applied as a layer to a substrate forms a coating layer having a refractive index of greater than about 1.5.

The recitation of numerical ranges by endpoints includes all numbers subsumed within the range (e.g. the range 1 to 10 includes 1, 1.5, 3.33, and 10).

As used in this specification and the appended claims, the singular forms “a”, “an”, and “the” include plural referents unless the content clearly dictates otherwise. Thus, for example, reference to a composition containing “a compound” includes a mixture of two or more compounds. As used in this specification and the appended claims, the term “or” is generally employed in its sense including “and/or” unless the content clearly indicates otherwise.

Unless otherwise indicated, all numbers expressing quantities of ingredients, measurements of properties such as contact angle and so forth as used in the specification and claims are to be understood to be modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as accurately as possible. Any numerical value, however, inherently contains certain errors necessarily resulting from the standard deviations found in their respective testing measurements.

The present invention is directed to antireflection materials used as a portion of optical displays (“displays”). The displays include various illuminated and non-illuminated displays panels wherein a combination of low surface energy (e.g. anti-soiling, stain resistant, oil and/or water repellency) and durability (e.g. abrasion resistance) is desired while maintaining optical clarity. The antireflection material functions to decrease glare and decrease transmission loss while improving durability and optical clarity.

Such displays include multi-character and especially multi-line multi-character displays such as liquid crystal displays (“LCDs”), plasma displays, front and rear projection displays, cathode ray tubes (“CRTs”), signage, as well as single-character or binary displays such as light emitting tubes (“LEDs”), signal lamps and switches. The light transmissive (i.e. exposed surface) substrate of such display panels may be referred to as a “lens.” The invention is particularly useful for displays having a viewing surface that is susceptible to damage.

The coating composition, and reactive product thereof, as well as the protective articles of the invention, can be employed in a variety of portable and non-portable information display articles. These articles include, but are not limited by, PDAs, LCD TV's (direct lit and edge lit), cell phones (including combination PDA/cell phones), touch sensitive screens, wrist watches, car navigation systems, global positioning systems, depth finders, calculators, electronic books, CD and DVD players, projection televisions screens, computer monitors, notebook computer displays, instrument gauges, instrument panel covers, signage such as graphic displays and the like. These devices can have planar viewing faces, or non-planar viewing faces such as slightly curved faces. The above listing of potential applications should not be construed to unduly limit the invention.

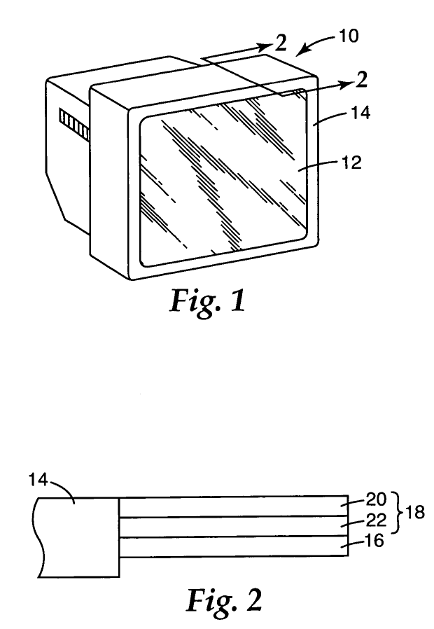

Referring now to FIG. 1, a perspective view of an article, here a computer monitor 10, is illustrated according to one preferred embodiment as having an optical display 12 coupled within a housing 14. The optical display 12 is a substantially transparent material having optically enhancing properties through which a user can view text, graphics or other displayed information.

As best shown in FIG. 2, the optical display 12 includes an antireflection film 18 coupled (coated) to an optical substrate 16. The antireflection film 18 has at least one layer of a high refraction index layer 22 and a low refractive index layer 20 coupled together such that the low refractive index layer 20 being positioned to be exposed to the atmosphere while the high refractive index layer 22 is positioned between the substrate 16 and low refractive index layer 20.

The optical substrate 16 preferably comprises an inorganic material, such as glass, or a polymeric organic material such as polyethylene terephthalate (“PET”), that are well known to those of ordinary skill in the optical display art. In addition, the substrate 16 may comprise a hybrid material, having both organic and inorganic components.

While not shown, other layers may be incorporated into the optical device, including, but not limited to, other hard coating layers, adhesive layers, and the like. Further, the antireflection material 18 may be applied directly to the substrate 16, or alternatively applied to a release layer of a transferable antireflection film and subsequently transferred from the release layer to the substrate using a heat press or photoradiation application technique.

The high refractive index layer 22 is a conventional carbon-based polymeric composition having a mono and multi-acrylate crosslinking system.

The low refractive index coating composition of the present invention used to form layer 20, in one aspect of the invention, is formed from the reaction product of a reactive fluoropolymer, a C═C double bond containing silane agent such as a multi-acrylate, 3-(trimethoxysilyl)propyl methacrylate and/or vinyltrimethoxysilane, and an optional multi-olefinic crosslinker. The reaction mechanism for forming the coating composition is described further below as Reaction Mechanism 1.

In another preferred approach, inorganic surface functionalized nanoparticles are added to the low refractive index composition 20 described in the preceding paragraphs to provide increased mechanical strength and scratch resistance to the low index coatings.

The low refractive index composition that is formed in any of the preferred approaches is then applied directly or indirectly to a substrate 16 of a display 12 to form a low refractive index portion 20 of an antireflection coating 18 on the article 10. With the invention, the article 10 has outstanding optical properties, including decreased glare and increased optical transmissivity. Further, the antireflection coating 18 has outstanding durability, as well as ink and stain repellency properties.

The ingredients for forming the various low refractive index compositions are summarized in the following paragraphs, followed by the reaction mechanism for forming the coatings according to each preferred approach.

Fluoropolymer

Fluoropolymer materials used in the low index coating may be described by broadly categorizing them into one of two basic classes. A first class includes those amorphous fluoropolymers comprising interpolymerized units derived from vinylidene fluoride (VDF) and hexafluoropropylene (HFP) and optionally tetrafluoroethylene (TFE) monomers. Examples of such are commercially available from 3M Company as Dyneon™ Fluoroelastomer FC 2145 and FT 2430. Additional amorphous fluoropolymers contemplated by this invention are for example VDF-chlorotrifluoroethylene copolymers, commercially known as Kel-F™ 3700, available from 3M Company. As used herein, amorphous fluoropolymers are materials that contain essentially no crystallinity or possess no significant melting point as determined for example by differential scanning caloriometry (DSC). For the purpose of this discussion, a copolymer is defined as a polymeric material resulting from the simultaneous polymerization of two or more dissimilar monomers and a homopolymer is a polymeric material resulting from the polymerization of a single monomer.

The second significant class of fluoropolymers useful in this invention are those homo and copolymers based on fluorinated monomers such as TFE or VDF which do contain a crystalline melting point such as polyvinylidene fluoride (PVDF, available commercially from 3M Company as Dyneon™ PVDF, or more preferable thermoplastic copolymers of TFE such as those based on the crystalline microstructure of TFE-HFP-VDF. Examples of such polymers are those available from 3M under the trade name Dyneon™ Fluoroplastic THV™ 200.

A general description and preparation of these classes of fluoropolymers can be found in Encyclopedia Chemical Technology, Fluorocarbon Elastomers, Kirk-Othmer (1993), or in Modern Fluoropolymers, J. Scheirs Ed, (1997), J Wiley Science, Chapters 2, 13, and 32. (ISBN 0-471-97055-7).

The preferred fluoropolymers are copolymers formed from the constituent monomers known as tetrafluoroethylene (“TFE”), hexafluoropropylene (“HFP”), and vinylidene fluoride (“VDF,” “VF2,”). The monomer structures for these constituents are shown below:
TFE: CF₂═CF₂ (1)
VDF: CH₂═CF₂ (2)
HFP: CF₂═CF—CF₃ (3)

The preferred fluoropolymer consists of at least two of the constituent monomers (HFP and VDF), and more preferably all three of the constituents monomers in varying molar amounts. Additional monomers not depicted in (1), (2) or (3) but also useful in the present invention include perfluorovinyl ether monomers of the general structure CF₂═CF—OR_{f}, wherein R_{f} can be a branched or linear perfluoroalkyl radicals of 1-8 carbons and can itself contain additional heteroatoms such as oxygen. Specific examples are perfluoromethyl vinyl ether, perfluoropropyl vinyl ethers, perfluoro(3-methoxy-propyl) vinyl ether. Additional examples are found in Worm (WO 00/12574), assigned to 3M, and in Carlson (U.S. Pat. No. 5,214,100).

For the purposes of the present invention, crystalline copolymers with all three constituent monomers shall be hereinafter referred to as THV, while amorphous copolymers consisting of VDF-HFP and optionally TFE is hereinafter referred to as FKM, or FKM elastomers as denoted in ASTM D 1418. THV and FKM elastomers have the general formula (4): 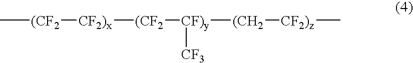
wherein x, y and z are expressed as molar percentages.

For fluorothermoplastics materials (crystalline) such as THV, x is greater than zero and the molar amount of y is typically less than about 15 molar percent. One commercially available form of THV contemplated for use in the present invention is Dyneon™ Fluorothermoplastic THV™ 220, a polymer that is manufactured by Dyneon LLC, of Saint Paul Minn. Other useful fluorothermoplastics meeting these criteria and commercially available, for example, from Dyneon LLC, Saint Paul Minn., are sold under the trade names THV™ 200, THV™ 500, and THV™ 800. THV™ 200 is most preferred since it is readily soluble in common organic solvents such as MEK and this facilitates coating and processing, however this is a choice born out of preferred coating behavior and not a limitation of the material used a low refractive index coating.

In addition, other fluoroplastic materials not specifically falling under the criteria of the previous paragraph are also contemplated by the present invention. For example, PVDF-containing fluoroplastic materials having very low molar levels of HFP are also contemplated by the present invention and are sold under the trade name Dyneon™ PVDF 6010 or 3100, available from Dyneon LLC, of St. Paul, Minn.; and Kynar™ 740, 2800, 9301, available from Elf Atochem North America Inc. Further, other fluoroplastic materials are specifically contemplated wherein x is zero and wherein y is between about 0 and 18 percent. Optionally the microstructure shown in (4) can also contain additional non-fluorinated monomers such as ethylene, propylene, or butylene. Examples of which are commercially available as Dyneon™ ETFE and Dyneon™ HTE fluoroplastics.

For fluoroelastomers compositions (amorphous) useful in the present invention, x can be zero so long as the molar percentage of y is sufficiently high (typically greater than about 18 molar percent) to render the microstructure amorphous. One example of a commercially available elastomeric compound of this type is available from Dyneon LLC of St. Paul, Minn., under the trade name Dyneon™ Fluoroelastomer FC 2145.

Additional fluoroelastomer compositions useful in the present invention exist where x is greater than zero. Such materials are often referred to as elastomeric TFE containing terpolymers. One example of a commercially available elastomeric compound of this type is available from Dyneon LLC of St. Paul, Minn., and is sold under the trade name Dyneon™ Fluoroelastomer FT 2430.

In addition, other fluorelastomeric compositions not classified under the preceding paragraphs are also useful in the present invention. For example, propylene-containing fluoroelastomers are a class useful in this invention. Examples of propylene-containing fluoroelastomers known as base resistant elastomers (“BRE”) and are commercially available from Dyneon under the trade name Dyneon™ BRE 7200. available from 3M Company of St. Paul, Minn. Other examples of TFE-propylene copolymer can also be used are commercially available under the tradename Aflaf™, available from Asahi Glass Company of Charlotte, N.C.

In one preferred approach, these polymer compositions further comprise reactive functionality such as halogen-containing cure site monomers (“CSM”) and/or halogenated endgroups, which are interpolymerized into the polymer microstructure using numerous techniques known in the art. These halogen groups provide reactivity towards the other components of coating mixture and facilitate the formation of the polymer network. Useful halogen-containing monomers are well known in the art and typical examples are found in U.S. Pat. No. 4,214,060 to Apotheker et al., European Patent No. EP398241 to Moore, and European Patent No. EP407937B1 to Vincenzo et al.

In addition to halogen containing cure site monomers, it is conceivable to incorporate nitrile-containing cure site monomers in the fluoropolymer microstructure. Such CSM's are particularly useful when the polymers are perfluorinated, i.e. contain no VDF or other hydrogen containing monomers. Specific nitrile-containing CSM's contemplated by this invention are described in Grootaret et al. (U.S. Pat. No. 6,720,360, assigned to 3M).

Optionally halogen cure sites can be introduced into the polymer microstructure via the judicious use of halogenated chain transfer agents which produce fluoropolymer chain ends that contain reactive halogen endgroups. Such chain transfer agents (“CTA”) are well known in the literature and typical examples are: Br—CF₂CF₂—Br, CF₂Br₂, CF₂I₂, CH₂I₂. Other typical examples are found in U.S. Pat. No. 4,000,356 to Weisgerber. Whether the halogen is incorporated into the polymer microstructure by means of a CSM or CTA agent or both is not particularly relevant as both result in a fluoropolymer which is more reactive towards UV crosslinking and coreaction with other components of the network such as the acrylates. An advantage to use of cure site monomers in forming the co-crosslinked network, as opposed to a dehydrofluorination approach (discussed below), is that the optical clarity of the formed polymer layer is not compromised since the reaction of the acrylate and the fluoropolymer does not rely on unsaturation in the polymer backbone in order to react. Thus, a bromo-containing fluoroelastomer such as Dyneon™ E-15472, E-18905, or E-18402 available from Dyneon LLC of St. Paul, Minn., may be used in conjunction with, or in place of, THV or FKM as the fluoropolymer.

In another embodiment the fluoropolymer microstructure is first dehydrofluorinated by any method that will provide sufficient carbon-carbon unsaturation of the fluoropolymer to create increased bond strength between the fluoropolymer and a hydrocarbon substrate or layer. The dehydrofluorination process is a well-known process to induced unsaturation and it is used most commonly for the ionic crosslinking of fluoroelastomers by nucleophiles such as diphenols and diamines. This reaction is an inherent property of VDF containing elastomers or THV. A descriptions can be found in The Chemistry of Fluorocarbon Elastomer, A.L. Logothetis, Prog. Polymer Science (1989), 14, 251. Furthermore, such a reaction is also possible with primary and secondary aliphatic monofunctional amines and will produce a DHF-fluoropolymer with a pendent amine side group. However, such a DHF reaction is not possible in polymers which do not contain VDF units since they lack the ability to lose HF by such reagents.

In addition to the main types of fluoropolymers useful in the context of this invention, there is a third special case involving the use of perfluoropolymers or ethylene containing fluoropolymers which are exempt form the DHF addition reaction described above but nonetheless are reactive photochemically with amines to produce low index fluoropolymer coatings. Examples of such are copolymers of TFE with HFP or perfluorovinyl ethers, or 2,2-bistrifluoromethyl-4,5-difluoro 1,3 dioxole. Such perfluoropolymers are commercially available as Dyneon™ Perfluoroelastomer, DuPont Kalrez™ or DuPont Teflon™ AF. Examples of ethylene containing fluoropolymers are known as Dyneon™ HTE or Dyneon™, ETFE copolymers. Such polymers are described in the above-mentioned reference of Scheirs Chapters 15, 19 and 22. Although these polymers are not readily soluble in typical organic solvents, they can be solubilized in such perfluoroinated solvents such as HFE 7100 and HFE 7200 (available from 3M Company, St. Paul, Minn.). These types of fluoropolymers are not easily bonded to other polymers or substrates. However the work of Jing et al, in U.S. Pat. Nos. 6,685,793 and 6,630,047, teaches methods where by such materials can be photochemcially grafted and bonded to other substrates in the presence of amines. However in these particular applications the concept of solution coatings and co-crosslinking in the presence of multifunctional acrylates is not contemplated.

Of course, as one of ordinary skill recognizes, other fluoropolymers and fluoroelastomers not specifically listed above may be available for use in the present invention. As such, the above listings should not be considered limiting, but merely indicative of the wide variety of commercially available products that can be utilized.

The compatible organic solvent that is utilized in the preferred embodiments of the present invention is methyl ethyl ketone (“MEK”). However, other organic solvents including fluorinated solvents may also be utilized, as well as mixtures of compatible organic solvents, and still fall within the spirit and scope of the present invention. For example, other organic solvents contemplated include acetone, cyclohexanone, methyl isobutyl ketone (“MIBK”), methyl amyl ketone (“MAK”), tetrahydrofuran (“THF”), methyl acetate, isopropyl alcohol (“IPA”), and mixtures thereof, may also be utilized.

C═C Double Bond Containing Silane Ester Agent

The preferred photograftable resins are those having a C═C double-bond containing silane ester agents. Example of preferred C═C double bond containing silane ester agents include 3-(trimethoxysilyl) propyl methacrylate (used under the trade designation “A-174” and vinyltrimethoxy silane (“VS”). However, other vinyl silane compounds or oligomers are also contemplated.

The unique feature of these agents is the ability of these crosslinkers to first react with the fluoropolymer backbone to form a silyl-grafted fluoropolymer that can be subsequently crosslinked to another pendent silyl group via a silane condensation reaction in the presence of moisture.

Nucleophilic amino groups such as primary or secondary aminosilane esters readily react with electrophilic double bond such as multiacrylates to undergo Michael addition even at room temperature as described the following reaction scheme.

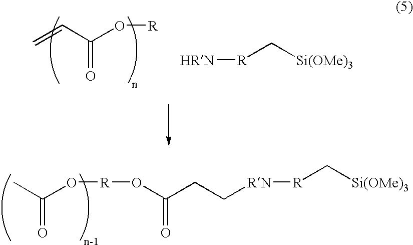

Such a reaction scheme forms alkoxysilyl containing mono- or multiacrylates. Available multiacrylates and aminosilane esters for the formation of the desired alkoxysilyl-containing acrylate and multiacrylate are generally formed according the following reaction scheme:

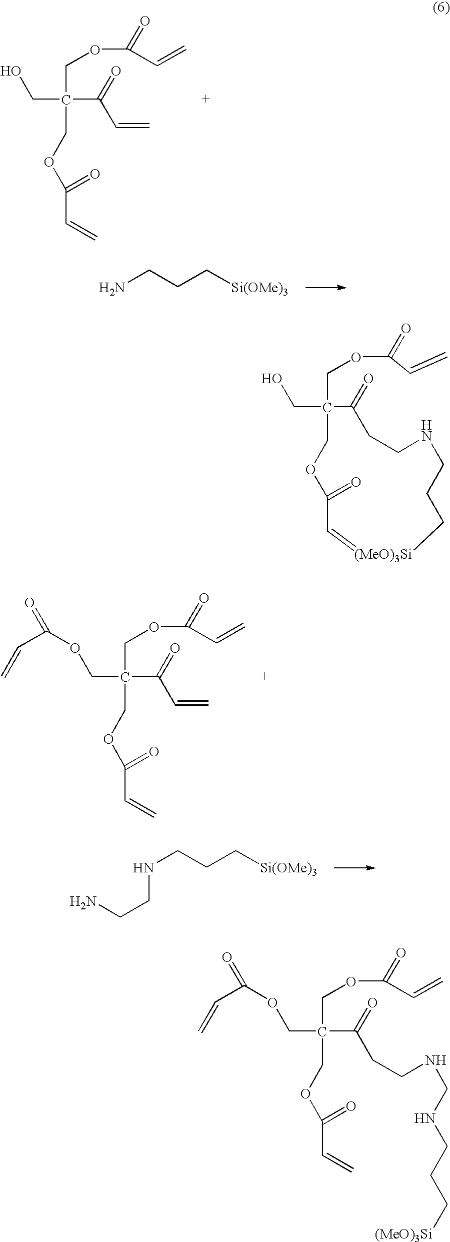

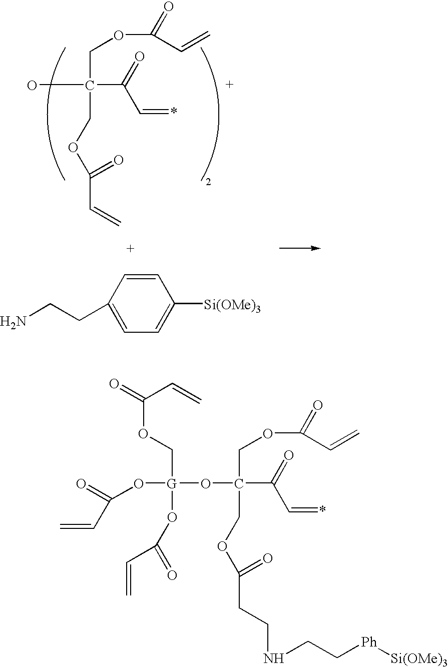

Suitable aminosilane esters for making the desired alkoxysilyl-containing multiacrylate can be formed from amino-substituted organosilane ester or ester equivalent that bear on the silicon atom at least one ester or ester equivalent group, preferably 2, or more preferably 3 groups. Ester equivalents are well known to those skilled in the art and include compounds such as silane amides (RNR′Si), silane alkanoates (RC(O)OSi), Si—O—Si, SiN(R)—Si, SiSR and RCONR′Si. These ester equivalents may also be cyclic such as those derived from ethylene glycol, ethanolamine, ethylenediamine and their amides. R and R′ are defined as in the “ester equivalent” definition in the Summary. Another such cyclic example of an ester equivalent (7):

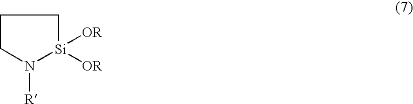

In this cyclic example R′ is as defined in the preceding sentence except that it may not be aryl. 3-aminopropyl alkoxysilanes are well known to cyclize on heating and these RNHSi compounds would be useful in this invention. Preferably the amino-substituted organosilane ester or ester equivalent has ester groups such as methoxy that are easily volatilized as methanol so as to avoid leaving residue at the interface that may interfere with bonding. The amino-substituted organosilane must have at least one ester equivalent; for example, it may be a trialkoxysilane. For example, the amino-substituted organosilane may have the formula (Z2N—L—SiX′X″X″′), where Z is hydrogen, alkyl, or substituted aryl or alkyl including amino-substituted alkyl; where L is a divalent straight chain C1-12 alkylene or may comprise a C3-8 cycloalkylene, 3-8 membered ring heterocycloalkylene, C2-12 alkenylene, C4-8 cycloalkenylene, 3-8 membered ring heterocycloalkenylene or heteroarylene unit. L, may be divalent aromatic or may be interrupted by one or more divalent aromatic groups or heteroatomic groups. The aromatic group may include a heteroaromatic. The heteroatom is preferably nitrogen, sulfur or oxygen. L is optionally substituted with C1-4 alkyl, C2-4 alkenyl, C2-4 alkynyl, C1-4 alkoxy, amino, C3-6 cycloalkyl, 3-6 membered heterocycloalkyl, monocyclic aryl, 5-6 membered ring heteroaryl, C1-4 alkylcarbonyloxy, C1-4 alkyloxycarbonyl, C1-4 alkylcarbonyl, formyl, C1-4 alkylcarbonylamino, or C1-4 aminocarbonyl. L is further optionally interrupted by —O—, —S—, —N(Rc)—, —N(Rc)—C(O)—, —N(Rc)-C(O)—O—, —O—C(O)—N(Rc)—, —N(Rc)—C(O)—N(Rd)—, —O—C(O)—, —C(O)—O—, or —O—C(O)—O—. Each of Rc and Rd, independently, is hydrogen, alkyl, alkenyl, alkynyl, alkoxyalkyl, aminoalkyl (primary, secondary or tertiary), or haloalkyl; and each of X′, X″ and X″′ is a C1-18 alkyl, halogen, C1-8 alkoxy, C1-8 alkylcarbonyloxy, or amino group, with the proviso that at least one of X′, X″, and X″′ is a labile group. Further, any two or all of X′, X″ and X″′ may be joined through a covalent bond. The amino group may be an alkylamino group.

Examples of amino-substituted organosilanes include 3-aminopropyltrimethoxysilane (SILQUEST A-1110); 3-aminopropyltriethoxysilane (SILQUEST A-1100); 3-(2-aminoethyl)aminopropyltrimethoxysilane (SILQUEST A-1120); SILQUEST A-1130, (aminoethylaminomethyl)phenethyltrimethoxysilane; (aminoethylaminomethyl)phenethyltriethoxysilane; N-(2-aminoethyl)-3-aminopropylmethyldimethoxysilane (SILQUEST A-2120), bis-(γ-triethoxysilylpropyl) amine (SILQUEST A-1170); N-(2-aminoethyl)-3-aminopropyltributoxysilane; 6-(aminohexylaminopropyl)trimethoxysilane; 4-aminobutyltrimethoxysilane; 4-aminobutyltriethoxysilane; p-(2-aminoethyl)phenyltrimethoxysilane; 3-aminopropyltris(methoxyethoxyethoxy)silane; 3-aminopropylmethyldiethoxysilane; oligomeric aminosilanes such as DYNASYLAN 1146, 3-(N-methylamino)propyltrimethoxysilane; N-(2-aminoethyl)-3-aminopropylmethyldimethoxysilane; N-(2-aminoethyl)-3-aminopropylmethyldiethoxysilane; N-(2-aminoethyl)-3-aminopropyltrimethoxysilane; N-(2-aminoethyl)-3-aminopropyltriethoxysilane; 3-aminopropylmethyldiethoxysilane; 3-aminopropylmethyldimethoxysilane; 3-aminopropyldimethylmethoxysilane; 3-aminopropyldimethylethoxysilane; 4-aminophenyltrimethoxy silane; 2,2-dimethoxy-1-aza-2-silacyclopentane-1-ethanamine (8); 2,2-diethoxy-1-aza-2-silacyclopentane-1-ethanamine (9); 2,2-diethoxy-1-aza-2-silacyclopentane (10); and 2,2-dimethoxy-1-aza-2-silacyclopentane (11).

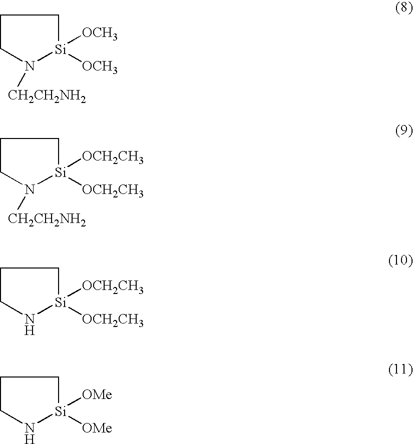

Additional “precursor” compounds such as a bis-silyl urea [RO)₃Si(CH₂)NR]₂C═O are also examples of amino-substituted organosilane ester or ester equivalents that liberate amine by first dissociating thermally.

The amino-substituted organosilane ester or ester equivalent is preferably introduced diluted in an organic solvent such as ethyl acetate or methanol or methyl acetate. One preferred amino-substituted organosilane ester or ester equivalent is 3-aminopropyl methoxy silane (H₂N—(CH₂)₃—Si(OMe)₃), or its oligomers.

One such oligomer is Silquest A-1106, manufactured by Osi Specialties (GE Silicones) of Paris, France. The amino-substituted organosilane ester or ester equivalent reacts with the fluoropolymer in a process described further below to provide pendent siloxy groups that are available for forming siloxane bonds with other antireflection layers to improve interfacial adhesion between the layers. The coupling agent thus acts as an adhesion promoter between the layers.

Suitable multiacrylates for making alkoxysilyl containing mono or multiacrylates are preferably based on a multi-olefinic crosslinking agent. More preferably, the multi-olefinic crosslinker in one that can be homopolymerizable. Most preferably, the multi-olefinic crosslinker is a multi-acrylate crosslinker.

Useful crosslinking acrylate agents include, for example, poly (meth)acryl monomers selected from the group consisting of (a) di(meth)acryl containing compounds such as 1,3-butylene glycol diacrylate, 1,4-butanediol diacrylate, 1,6-hexanediol diacrylate, 1,6-hexanediol monoacrylate monomethacrylate, ethylene glycol diacrylate, alkoxylated aliphatic diacrylate, alkoxylated cyclohexane dimethanol diacrylate, alkoxylated hexanediol diacrylate, alkoxylated neopentyl glycol diacrylate, caprolactone modified neopentylglycol hydroxypivalate diacrylate, caprolactone modified neopentylglycol hydroxypivalate diacrylate, cyclohexanedimethanol diacrylate, diethylene glycol diacrylate, dipropylene glycol diacrylate, ethoxylated (10) bisphenol A diacrylate, ethoxylated (3) bisphenol A diacrylate, ethoxylated (30) bisphenol A diacrylate, ethoxylated (4) bisphenol A diacrylate, hydroxypivalaldehyde modified trimethylolpropane diacrylate, neopentyl glycol diacrylate, polyethylene glycol (200) diacrylate, polyethylene glycol (400) diacrylate, polyethylene glycol (600) diacrylate, propoxylated neopentyl glycol diacrylate, tetraethylene glycol diacrylate, tricyclodecanedimethanol diacrylate, triethylene glycol diacrylate, tripropylene glycol diacrylate; (b) tri(meth)acryl containing compounds such as glycerol triacrylate, trimethylolpropane triacrylate, ethoxylated triacrylates (e.g., ethoxylated (3) trimethylolpropane triacrylate, ethoxylated (6) trimethylolpropane triacrylate, ethoxylated (9) trimethylolpropane triacrylate, ethoxylated (20) trimethylolpropane triacrylate), pentaerythritol triacrylate, propoxylated triacrylates (e.g., propoxylated (3) glyceryl triacrylate, propoxylated (5.5) glyceryl triacrylate, propoxylated (3) trimethylolpropane triacrylate, propoxylated (6) trimethylolpropane triacrylate), trimethylolpropane triacrylate, tris(2-hydroxyethyl)isocyanurate triacrylate; (c) higher functionality (meth)acryl containing compounds such as ditrimethylolpropane tetraacrylate, dipentaerythritol pentaacrylate, ethoxylated (4) pentaerythritol tetraacrylate, pentaerythritol tetraacrylate, caprolactone modified dipentacrythritol hexaacrylate; (d) oligomeric (meth)acryl compounds such as, for example, urethane acrylates, polyester acrylates, epoxy acrylates; polyacrylamide analogues of the foregoing; and combinations thereof. Such compounds are widely available from vendors such as, for example, Sartomer Company, Exton, Pa.; UCB Chemicals Corporation, Smyrna, Ga.; and Aldrich Chemical Company, Milwaukee, Wis. Additional useful (meth)acrylate materials include hydantoin moiety-containing poly(meth)acrylates, for example, as described in U.S. Pat. No. 4,262,072 (Wendling et al.).

Multi-Olefinic Crosslinking Agent

The crosslinking agent of the present invention is based on a multi-olefinic crosslinking agent. More preferably, the multi-olefinic crosslinker in one that can be homopolymerizable. Most preferably, the multi-olefinic crosslinker is a multi-acrylate crosslinker.

Useful crosslinking acrylate agents include, for example, poly (meth)acryl monomers selected from the group consisting of (a) di(meth)acryl containing compounds such as 1,3-butylene glycol diacrylate, 1,4-butanediol diacrylate, 1,6-hexanediol diacrylate, 1,6-hexanediol monoacrylate monomethacrylate, ethylene glycol diacrylate, alkoxylated aliphatic diacrylate, alkoxylated cyclohexane dimethanol diacrylate, alkoxylated hexanediol diacrylate, alkoxylated neopentyl glycol diacrylate, caprolactone modified neopentylglycol hydroxypivalate diacrylate, caprolactone modified neopentylglycol hydroxypivalate diacrylate, cyclohexanedimethanol diacrylate, diethylene glycol diacrylate, dipropylene glycol diacrylate, ethoxylated (10) bisphenol A diacrylate, ethoxylated (3) bisphenol A diacrylate, ethoxylated (30) bisphenol A diacrylate, ethoxylated (4) bisphenol A diacrylate, hydroxypivalaldehyde modified trimethylolpropane diacrylate, neopentyl glycol diacrylate, polyethylene glycol (200) diacrylate, polyethylene glycol (400) diacrylate, polyethylene glycol (600) diacrylate, propoxylated neopentyl glycol diacrylate, tetraethylene glycol diacrylate, tricyclodecanedimethanol diacrylate, triethylene glycol diacrylate, tripropylene glycol diacrylate; (b) tri(meth)acryl containing compounds such as glycerol triacrylate, trimethylolpropane triacrylate, ethoxylated triacrylates (e.g., ethoxylated (3) trimethylolpropane triacrylate, ethoxylated (6) trimethylolpropane triacrylate, ethoxylated (9) trimethylolipropane triacrylate, ethoxylated (20) trimethylolpropane triacrylate), pentaerythritol triacrylate, propoxylated triacrylates (e.g., propoxylated (3) glyceryl triacrylate, propoxylated (5.5) glyceryl triacrylate, propoxylated (3) trimethylolpropane triacrylate, propoxylated (6) trimethylolpropane triacrylate), trimethylolpropane triacrylate, tris(2-hydroxyethyl)isocyanurate triacrylate; (c) higher functionality (meth)acryl containing compounds such as ditrimethylolpropane tetraacrylate, dipentaerythritol pentaacrylate, ethoxylated (4) pentaerythritol tetraacrylate, pentaerythritol tetraacrylate, caprolactone modified dipentaerythritol hexaacrylate; (d) oligomeric (meth)acryl compounds such as, for example, urethane acrylates, polyester acrylates, epoxy acrylates; polyacrylamide analogues of the foregoing; and combinations thereof. Such compounds are widely available from vendors such as, for example, Sartomer Company, Exton, Pa.; UCB Chemicals Corporation, Smyrna, Ga.; and Aldrich Chemical Company, Milwaukee, Wis. Additional useful (meth)acrylate materials include hydantoin moiety-containing poly(meth)acrylates, for example, as described in U.S. Pat. No. 4,262,072, to Wendling et al.

A preferred crosslinking agent comprises at least three (meth)acrylate functional groups. Preferred commercially available crosslinking agents include those available from Sartomer Company, Exton, Pa. such as trimethylolpropane triacrylate (TMPTA) available under the trade designation “SR351”, pentaerythritol tri/tetraacrylate (PETA) available under the trade designation “SR444” or “SR494”, and dipentaerythritol hexaacrylate available under the trade designation “SR399.” Further, mixtures of multifunctional and lower functional acrylates (monofunctional acrylates), such as a mixture of TMPTA and MMA (methyl methacrylate), may also be utilized.

Other preferred crosslinkers that may be utilized in the present invention include fluorinated acrylates exemplified by perfluoropolyether acrylates. These perfluoropolyether acrylates are based on monofunctional acrylate and/or multi-acrylate derivatives of hexafluoropropylene oxide (“HFPO”) and may be used as the sole crosslinker, or more preferably, in conjunction with TMPTA or PETA.

Many types of olefinic compounds such as divinyl benzene or 1,7-cotadiene and others like might be expected to behave as crosslinkers under the present conditions.

Perfuoropolyether mono- or multi-acrylates were also used to interact with the fluoropolymers, especially bromo-containing fluoropolymers, for further improving surface properties and lowering refractive indices. Such acrylates provide hydro and olephobicity properties typical of fluorochemical surfaces to provide anti-soiling, release and lubricative treatments for a wide range of substrates without affecting optical properties.

As used in the examples, “HFPO-” refers to the end group F{CF(CF₃)CF₂O}aCF(CF₃)— wherein “a” averages about 6.3, with an average molecular weight of 1,211 g/mol, and which can be prepared according to the method reported in U.S. Pat. No. 3,250,808 (Moore et al.), the disclosure of which is incorporated herein by reference, with purification by fractional distillation.

Surface Modified Nanoparticles

The mechanical durability of the resultant low refractive index layers 20 can be enhanced by the introduction of surface modified inorganic particles.

These inorganic particles can have a substantially monodisperse size distribution or a polymodal distribution obtained by blending two or more substantially monodisperse distributions. The inorganic oxide particles are typically non-aggregated (substantially discrete), as aggregation can result in precipitation of the inorganic oxide particles or gelation of the hardcoat. The inorganic oxide particles are typically colloidal in size, having an average particle diameter of 5 nanometers to 100 nanometers. These size ranges facilitate dispersion of the inorganic oxide particles into the binder resin and provide ceramers with desirable surface properties and optical clarity. The average particle size of the inorganic oxide particles can be measured using transmission electron microscopy to count the number of inorganic oxide particles of a given diameter. Inorganic oxide particles include colloidal silica, colloidal titania, colloidal alumina, colloidal zirconia, colloidal vanadia, colloidal chromia, colloidal iron oxide, colloidal antimony oxide, colloidal tin oxide, and mixtures thereof. Most preferably, the particles are formed of silicon dioxide (SiO₂).

The surface particles are modified with polymer coatings designed to have alkyl or fluoroinated alkyl groups, and mixtures thereof, that have reactive functionality towards the fluoropolymer. Such functionalities include mercaptan, vinyl, acrylate and others believed to enhance the interaction between the inorganic particles and low index fluoropolymers, especially those containing chloro, bromo, iodo or alkoxysilane cure site monomers. Specific surface modifying agents contemplated by this invention include but are not limited to 3-methacryloxypropyltrimethoxysilane A174 OSI Specialties Chemical), vinyl trialkoxy silanes such as trimethoxy and triethoxy silane and hexamethydisilizane (available from Aldrich Co).

These vinylidene fluoride containing fluoropolymers are known to enable grafting with chemical species having nucleophilic groups such as —NH₂, —SH, and —OH via dehydrofluorination and Michael addition processes.

Photoinitiators and Additives

To facilitate curing, polymerizable compositions according to the present invention may further comprise at least one free-radical photoinitiator. Typically, if such an initiator photoinitiator is present, it comprises less than about 10 percent by weight, more typically less than about 5 percent of the polymerizable composition, based on the total weight of the polymerizable composition.

Free-radical curing techniques are well known in the art and include, for example, thermal curing methods as well as radiation curing methods such as electron beam or ultraviolet radiation. Further details concerning free radical thermal and photopolymerization techniques may be found in, for example, U.S. Pat. Nos. 4,654,233 (Grant et al.); 4,855,184 (Klun et al.); and 6,224,949 (Wright et al.).

Useful free-radical photoinitiators include, for example, those known as useful in the UV cure of acrylate polymers. Such initiators include benzophenone and its derivatives; benzoin, alpha-methylbenzoin, alpha-phenylbenzoin, alpha-allylbenzoin, alpha-benzylbenzoin; benzoin ethers such as benzil dimethyl ketal (commercially available under the trade designation “IRGACURE 651” from Ciba Specialty Chemicals Corporation of Tarrytown, N.Y.), benzoin methyl ether, benzoin ethyl ether, benzoin n-butyl ether; acetophenone and its derivatives such as 2-hydroxy-2-meibyl-1-phenyl-1-propanone (commercially available under the trade designation “DAROCUR 1173” from Ciba Specialty Chemicals Corporation) and 1-hydroxycyclohexyl phenyl ketone (commercially available under the trade designation “IRGACURE 184”, also from Ciba Specialty Chemicals Corporation); 2-methyl-1-[4-(methylthio)phenyl]-2-(4-morpholinyl)-1-propanone commercially available under the trade designation “IRGACURE 907”, also from Ciba Specialty Chemicals Corporation); 2-benzyl-2-(dimethylamino)-1-[4-(4-morpholinyl)phenyl]-1-butanone commercially available under the trade designation “IRGACURE 369” from Ciba Specialty Chemicals Corporation); aromatic ketones such as benzophenone and its derivatives and anthraquinone and its derivatives; onium salts such as diazonium salts, iodonium salts, sulfonium salts, titanium complexes such as, for example, that which is commercially available under the trade designation “CGI 784 DC”, also from Ciba Specialty Chemicals Corporation); halomethylnitrobenzenes; and mono- and bis-acylphosphines such as those available from Ciba Specialty Chemicals Corporation under the trade designations “IRGACURE 1700”, “IRGACURE 1800”, “IRGACURE 1850”, “IRGACURE 819” “IRGACURE 2005”, “IRGACURE 2010”, “IRGACURE 2020” and “DAROCUR 4265”. Combinations of two or more photoinitiators may be used. Further, sensitizers such as 2-isopropyl thioxanthone, commercially available from First Chemical Corporation, Pascagoula, Miss., may be used in conjunction with photoinitiator(s) such as “IRGACURE 369”.

More preferably, the initiators used in the present invention are either “DAROCURE 1173” or “ESACURE® KB-1”, a benzildimethylketal photoinitiator available from Lamberti S.p.A of Gallarate, Spain.

Alternatively, or in conjunction herewith, the use of thermal initiators may also be incorporated into the reaction mixture. Useful free-radical thermal initiators include, for example, azo, peroxide, persulfate, and redox initiators, and combinations thereof.

Those skilled in the art appreciate that the coating compositions can contain other optional adjuvants, such as, surfactants, antistatic agents (e.g., conductive polymers), leveling agents, photosensitizers, ultraviolet (“UV”) absorbers, stabilizers, antioxidants, lubricants, pigments, dyes, plasticizers, suspending agents and the like.

The reaction mechanism for forming the low refractive index composition for the preferred approach (REACTION MECHANISM 1) is described in further detail below:

### Reaction Mechanism 1

In an alternative preferred approach, the cured site fluoropolymers described above could first be thermally or photochemically photografted with C═C double-bond containing silane reagents such as 3-(trimethoxysilyl) propyl methacrylate, vinyltrimethoxy silane, or other vinyl silane. An optional multi-olefinic (and more preferably a multifunctional (meth)acrylate crosslinker) is then added to the resultant fluoropolymer solution, and the mixture irradiated to form the low refractive index composition.

Step 1: Introduction of C═C Containing Silane Reagent and Multi-Olefinic Crosslinker to Fluoropolymer and Subsequent Application to a Substrate Material

In Reaction Mechanism 1, a fluoropolymer as described above is first dissolved in a compatible organic solvent. Preferably, the solution is about 10% by weight fluoropolymer and 90% by weight organic solvent. Preferably, the fluoropolymer has a plurality of cure site monomers, and more preferably the fluoropolymer has a plurality of bromo-, iodo-, and chloro-containing cure sites.

In addition, surface modified nanoparticles as described above may optionally be added to the fluoropolymer solution in amounts not exceeding about 5-10% by weight of the overall low refractive index composition.

The compatible organic solvent that is utilized in the preferred embodiments of the present invention is methyl ethyl ketone (“MEK”). However, other organic solvents may also be utilized, as well as mixtures of compatible organic solvents, and still fall within the spirit and scope of the present invention. For example, other organic solvents contemplated include acetone, cyclohexanoe, methyl isobutyl ketone (“MIBK”), methyl amyl ketone (“MAK”), tetrahydrofuran (“THF”), isopropyl alcohol (“IPA”), and mixtures thereof.

Next, a C═C double-bond containing silane reagent such as 3-(trimethoxysilyl) propyl methacrylate, vinyltrimethoxy silane, or other vinyl silanes, is added to the mixture.

A multi-olefinic crosslinker such as a C═C double bond containing multifunctional (meth)acrylate(including fluorinated acrylates) is then optionally (and preferably) introduced to the container having the fluoropolymer and C═C double bond containing silane reagent. The mixture is sealed in an airtight container and maintained at ambient conditions.

The resultant composition is then applied as a wet layer either (1) directly to an optical substrate or hardcoated optical substrate, or (2) to a high refractive index layer, or (3) to a release layer of a transferable film. The optical substrate could be glass or a polymeric material such as polyethylene terepthalate (PET).

Next, the wet layer is dried at between about 100 and 120 degrees Celsius for about ten minutes to form a dry layer (i.e. coated subject). Preferably, this is accomplished by introducing the substrate having the wet layer to an oven.

Step 2: Crosslinking Reaction

Next, the coated subject is irradiated with an ultraviolet light source to induce photocrosslinking of the C═C containing silane compound and the multifunctional (meth)acrylate to the fluoropolymer backbone. Preferably, the coated subject is subjected to ultraviolet radiation by H-bulb or by a 254-nanometer (nm) lamp in one or more passes along a conveyor belt to form the low refractive index layer 20. The UV processor preferably used is Fusion V, Model MC-6RQN with H-bulb, made by Fusion UV Systems, Inc. of Gaithersburg, Md.

Alternatively, the coated subject can be thermally crosslinked by applying heat and a suitable radical initiator such as a peroxide compound.

Two separate reaction mechanisms occur during this photocrosslinking step. First the C═C double bond containing silane reagent is photografted to the fluoropolymer backbone, preferably at the bromine containing cure sites, to form a silyl-modified fluoropolymer. The reaction mechanism for this reaction is shown below:

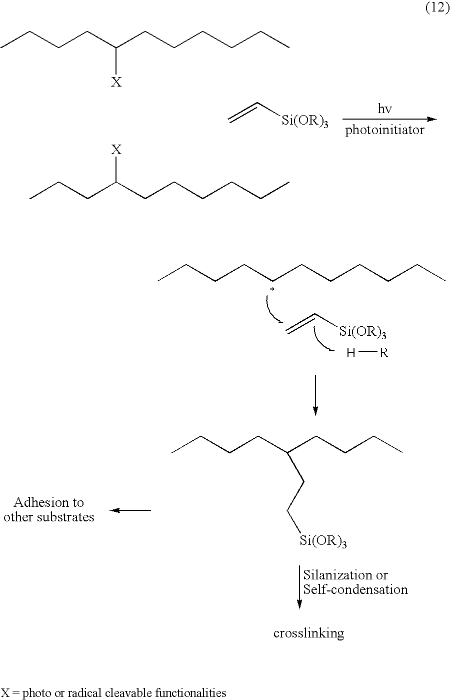

Such photografting can be made more efficient when the fluoropolymers have cure site monomers such as the afore-mentioned bromine, or also by iodine, chlorine and the like, which are more susceptible to being attacked by a radical species that hydrogen atoms of the fluoropolymer.

In addition, the optionally added multi-olefinic crosslinker crosslinks to the fluoropolymer backbone by the following reaction mechanism (13) (here, a multifunctional (meth)acrylate crosslinker is utilized as the multi-olefinic crosslinker).

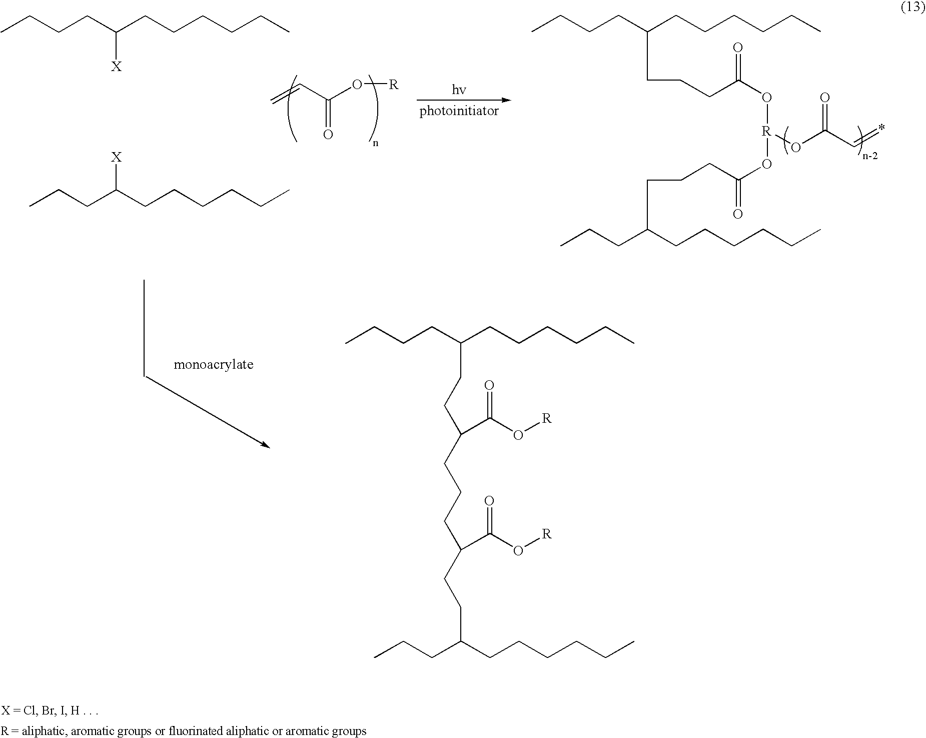

Alternatively, fluoropolymer crosslinking chemistry can be achieved by employing alkoxy-silyl containing multi-olefenic agents such as alkoxysilyl-containing multiacrylates.

The resultant composition has enhanced adhesion due to the presence of pendent silyl groups photografted onto the fluoropolymer backbone that can be further crosslinked, especially to other silyl containing surfaces such as high refractive index layers or hard coating layers, via silane condensation to form siloxane bonds. This enhances interfacial adhesion between the low refractive index layer and the adjacent layers, therein improving scratch resistance and durability of an antireflection film in which the low refractive index composition is used.

### EXAMPLES

The following paragraphs illustrate, via a specific set of example reactions and experimental methodologies, the improvements of each of the component steps for forming the low refractive index composition of the present invention.

A. Test Methods

1. Peel Strength

A peel strength was used to determine interfacial adhesion. To facilitate testing of the adhesion between the layers via a T-peel test, a thick film (20 mil (0.51 mm)) of THV 220 or FC 2145 was laminated onto the side of the films with the fluoropolymer coating in order to gain enough thickness for peel measurement. In some cases, a slight force was applied to the laminated sheet to keep a good surface contact. A strip of PTFE fiber sheet was inserted about 0.25 inch (0.64 mm) along the short edge of the 2 inch×3 inch (5.08 cm×7.62 cm) laminated sheet between the substrate surface and the fluoropolymer film to provide unbonded region to act as tabs for the peel test. The laminated sheet was then pressed at 200° C. for 2 minutes between heated platens of a Wabash Hydraulic Press (Wabash Metal Products Company, Inc., Hydraulic Division, Wabash, Ind.) and immediately transferred to a cold press. After cooling to room temperature by the cold press, the resulting sample was subjected to T-peel measurement.

Peel strengths of the laminated samples were determined following the test procedures described in ASTM D-1876 entitled “Standard Test Method for Peel Resistance of Adhesives,” more commonly known as the “T-peel” test. Peel data was generated using an INSTRON Model 1125 Tester (available from Instron Corp., Canton, Mass.) equipped with a Sintech Tester 20 (available from MTS Systems Corporation, Eden Prairie, Minn.). The INSTRON tester was operated at a cross-head speed of 4 inches/min (10.2 cm/min). Peel strength was calculated as the average load measured during the peel test and reported in pounds per inch (lb/inch) width (and N/cm) as an average of at least two samples.
2. Boiling Water Test

In the boiling water test, the coated sample was placed in boiling water for 2 hours. The sample was removed, and an inspection was performed on the sample to see if the low refractive index layer delaminated from the substrate.

B. Ingredients:

The ingredients used for forming the various coatings of this invention are summarized in the following paragraphs.

Dyneon ™ THV™ 220 Fluoroplastic (20 MFI, ASTM D 1238) is available as either a 30% solids latex grade under the trade name of Dyneon™ THV™ 220D Fluoroplastic dispersion, or as a pellet grade under the trade name of Dyneon™ THV™ 220G. Both are available from Dyneon LLC of St. Paul, Minn. In the case of Dyneon™ THV™ 220D, a coagulation step is necessary to isolate the polymer as a solid resin. The process for this is described below.

Dyneon™ FT 2430 and Dyneon™ FC 2145 fluoroelastomers are 70 wt % fluorine terpolymer and 66 wt % fluorine copolymer respectively, both available from Dyneon LLC of St. Paul, Minn. and were used as received.

Trimethylolpropane triacrylate SR 351 (“TMPTA”) and Di-Pentaerythritol tri acrylate (SR 399LV) were obtained from Sartomer Company of Exton, Pa. and used as received.

Acryloyl chloride was obtained from Sigma-Aldrich and used without further purification.

3-methacryloxypropyltrimethoxysilane available as A174 OSI Specialties Chemical was used as received.

3-aminopropyl triethoxy silane (3-APS) is available form Aldrich Chemical Milwaukee, Wis. and was used as received.

A1106-Silquest, manufactured by Osi Specialties (GE Silicones) of Paris, France.

“Darocur 1173” 2-hydroxy 2-methyl 1-phenyl propanone UV photoinitiator, and Irgacure™ 819 were obtained from Ciba Specialty Products, Terrytown, N.Y. and used as received.

“KB-1” benzyl dimethyl ketal UV photoinitiator was obtained from Sartomer Company of Exton, Pa. and was used as received.

Dowanol™, 1-methoxy-2-propanol was obtained from Sigma-Aldrich of Milwaukee, Wis. and used as received.

SR295, mixture of pentaerythritol tri and tetraacrylate, CN 120Z, Acrylated bisphenol A, SR 339, Phenoxyethyl acrylate, were obtained from Sartomer Chemical Company of Exton, Pa. and used as received.

(3-Acryloxypropyl)trimethoxysilane, was obtain from Gelest of Morrisville, Pa. and was used as received.

A1230, polyether silane was obtained from OSI Specialties and was used as received.

Buhler zirconia (ZrO2, was obtained from Buhler, Uzweil Switzerland and was used as received.

Oligomeric hexafluoropropylene oxide methyl ester (HFPO—C(O)OCH₃,) can be prepared according to the method reported in U.S. Pat. No. 3,250,808 (Moore et al.). The broad product distribution of oligomers obtained from this preparation can be fractionated according to the method described in U.S. patent application Ser. No. 10/331816, filed Dec. 30, 2002. This step yields the higher molecular weight distribution of oligomers used in this description wherein the number average degree of polymerization is about 6.3, and with an average molecular weight of 1,211 g/mol.
1. Coagulation of Dyneon™ THV™ 220D Latex:

The solid THV 220 resin derived from THV 220D latex can be obtained by freeze coagulation. In a typical procedure, 1-L of latex was placed in a plastic container and allowed to freeze at −18° C. for 16 hrs. The solids were allowed to thaw and the coagulated polymer was separated from the water phase by simple filtration. The solid polymer was than further divided into smaller pieces and washed 3-times with about 2 liters of hot water while being agitated. The polymer was collected and dried at 70-80° C. for 16 hours. Note whether THV 220D or THV 220G was used as the source of the preparation of the THV 220 solution, they are for the purposes of this application considered an equivalent.
2. Preparation of Hexafluoropropylene Oxide N-methyl-1,3-propanediamine Adduct

A 1-liter round-bottom flask was charged with 291.24 g (0.2405 mol) of FC-1 and 21.2 g (0.2405 mol) N-methyl-1,3-propanediamine, both at room temperature, resulting in a cloudy solution. The flask was swirled and the temperature of the mixture rose to 45° C., and to give a water-white liquid, which was heated overnight at 55° C. The product was then placed on a rotary evaporator at 75° C. and 28 inches of Hg vacuum to remove methanol, yielding 301.88 g of a viscous slightly yellow liquid, the hexafluoropropylene oxide N-methyl-1,3-propanediamine adduct.
3. Preparation of HFPO-acrylate-3

To a 250 ml roundbottom flask was charged with 4.48 g (15.2 mmoles, based on a nominal MW of 294) of trimethylolpropane triacrylate (TMPTA, Sartomer SR351), 4.45 g of tetrahydrofuran (THF), and 1.6 mg of phenothiazine and placed in an oil bath at 55 C. Next, in a 100 ml jar was dissolved 20 g (15.78 mmole, MW 1267.15) hexafluoropropylene oxide N-methyl-1,3-propanediamine adduct in 32 g THF. This solution was placed in a 60 ml dropping funnel with pressure equalizing sidearm, the jar rinsed with about 3 ml of THF, which was also added to the dropping funnel. The contents of the funnel were added over 38 minutes under an air atmosphere to the TMPTA/THF/phenothiazine mixture. The reaction was cloudy at first, but cleared at about 30 minutes. Twenty minutes after the addition was complete, the reaction flask was placed on a rotary evaporator at 45-55 rpm under 28 inches of vacuum to yield 24.38 g of a clear, viscous yellow liquid, that was characterized by NMR and HPLC/mass spectroscopy.
4. Preparation of Modified 20 nm Colloidal Silicon Dioxide Particles

15 g of 2327 (20 nm ammonium stabilized colloidal silica sol, 41% solids; Nalco, Naperville, Ill.) were placed in a 200 ml glass jar. A solution of 10 g of 1-methoxy-2-propanol (Aldrich) containing 0.57 g of vinyltrimethoxysilane (Gelest, Inc., Tullytown, Pa.) was prepared in a separate flask. The vinyltrimethoxysilane solution was added to the glass jar while the silica sol was stirred. The flask was then rinsed with an additional 5 ml of solvent and added to the stirred solution. After complete addition, the jar was capped and placed in an oven at 90 degrees Celsius for about 20 hours. The sol was then dried by exposure to gentle airflow at room temperature. The powdery white solid was collected and dispersed in 50 ml of THF solvent. 2.05 g of HMDS (excess) were slowly added to the THF silica sol, and, after addition, the jar was capped and placed in an ultrasonic bath for about 10 hours. Subsequently, the organic solvent was removed by a rotovap and the remaining white solid heated at 100 degrees Celsius overnight for further reaction and removal of volatile species. The resultant particles are noted below as vinyl modified/HMDS particles.

15 g of 2327 (20 nm ammonium stabilized colloidal silica sol, 41% solids; Nalco, Naperville, Ill.) were placed in a 200 ml glass jar. A solution of 10 g of 1-methoxy-2-propanol (Aldrich) containing 0.47 g of 3-(Trimethoxysilyl)propylmethacrylate (Gelest, Inc. of Tullytown, Pa.) was prepared in a separate flask. The 3-(Trimethoxysilyl)propylmethacrylate solution was added to the glass jar while the silica sol was stirred. The flask was then rinsed with an additional 5 ml of solvent and added to the stirred solution. After complete addition, the jar was capped and placed in an oven at 90 degrees Celsius for about 20 hours. The sol was then dried by exposure to gentle airflow at room temperature. The powdery white solid was collected and dispersed in 50 ml of THF solvent. 2.05 g of HMDS (excess) were slowly added to the THF silica sol, and, after addition, the jar was capped and placed in an ultrasonic bath for about 10 hours. Subsequently, the organic solvent was removed by a rotovap and the remaining white solid heated at 100 degrees Celsius overnight for further reaction and removal of volatile species. The resultant particles are noted below as A-174/HMDS particles.
5. Preparation of Modified Fumed Silica

The synthesis of partially acrylic-modified fumed SiO₂ was prepared by first making a sol of 2 g of SiO₂ (380 m²/g) and 100 ml of 1-methoxy-2-propanol (Aldrich) in a glass jar. 4 g of ammonium hydroxide (30% aqueous solution) and 20 g distilled water were then added slowly into the solution upon stirring. The mixture became a gel. A solution of 20 g of 1-methoxy-2-propanol (Aldrich) containing 0.2 g of 3-(Trimethoxysilyl)propylmethacrylate (Aldrich) was prepared in a separate flask.

The (trimethoxysilyl)propylmethacrylate solution was added to the glass jar while stirring. The flask was then rinsed with an additional 5-10 ml of the solvent and subsequently added to the stirred solution. After complete addition, the jar was capped and placed in an ultrasonic bath at 80 degrees Celsius for between 6 and 8 hours. The solution was then dried in a flow-through oven at room temperature. The powdery white solid was collected and dispersed into 50 ml of THF solvent. 2.05 g of HMDS (excess) was slowly added to the THF powder solution, and, after addition, the jar was capped and placed in an ultrasonic bath for about 10 hours. Subsequently, the organic solvent was removed by a rotovap and the white solid was heated at 100 degrees Celsius overnight for further reaction and removal of volatile species. The resultant particles are noted below as A-174/F-SiO₂ particles.
6. Preparation of Particles Modified by Vinyltriethoxysilane and HMDS

By ultrasonication, a sol containing 2 g of fumed SiO₂ (380 m²g) and 100 ml of 1-methoxy-2-propanol (Aldrich) was prepared in a glass jar. 4 g of ammonium hydroxide (30% aqueous solution) and 20 g distilled water were then added slowly into the solution with stirring. The mixture became a gel. A solution of 20 g of 1-methoxy-2-propanol (Aldrich) containing 0.2 g of vinyl triethoxysilane (Gelest, Inc. of Tullytown, Pa.) was prepared in a separate flask. The solution was added to the glass jar while stirring. The flask was then rinsed with an additional 5-10 ml of the solvent and added to the stirred solution. After complete addition, the jar was capped and placed in an ultrasonic bath at 80 degrees Celsius for between 6 to 8 hours. The solution was then dried in gentle airflow at room temperature. The powdery white solid was collected and dispersed into 50 ml of THF solvent. To the dispersed THF sol was slowly added 2.05 g of HMDS (excess). After addition, the jar was capped and placed in an ultrasonic bath for about 10 hours. Subsequently, the organic solvent was removed by a rotovap and the remaining white solid was heated at 100 degrees Celsius overnight for further reaction and removal of volatile species. The resultant particles are noted below as V/F-SiO₂ particles.
7. Description of PET Substrate (S1):

One preferred substrate material is polyethylene terephthalate (PET) film obtained from e.i. DuPont de Nemours and Company of Wilmington, Del. under the trade designation “Melinex 618”, and having a thickness of 5.0 mils and a primed surface. Referred to in the examples herein as substrate S1.
8. Description of the Hardcoated Substrate (S2):

Typically, the hardcoat is formed by coating a curable liquid ceramer composition onto a substrate, in this case primed PET substrate (S1), and curing the composition in situ to form a hardened film (or hardcoated substrate (S2). Suitable coating methods include those previously described for application of the fluorochemical surface layer. Further, details concerning hardcoats can be found in U.S. Pat. Nos. 6,132,861 to Kang et al., 6,238,798 to Kang et al., 6,245,833 to Kang et al., and 6,299,799 to Craig et al. A hardcoat composition that was substantially the same as Example 3 of U.S. Pat. No. 6,299,799 was coated onto the primed surface of S1 and cured in a UV chamber having less than 50 parts per million (ppm) oxygen. The UV chamber was equipped with a 600 watt H-type bulb from Fusion UV systems of Gaithersburg, Md., operating at full power. The hard coat was applied to S1 with a metered, precision die coating process. The hard coat was diluted in IPA to 30 weight percent solids and coated onto the 5-mil PET backing to achieve a dry thickness of 5 microns. A flow meter was used to monitor and set the flow rate of the material from a pressurized container. The flow rate was adjusted by changing the air pressure inside the sealed container which forces liquid out through a tube, through a filter, the flow meter and then through the die. The dried and cured film (S2) was wound on a take up roll and used as the input backing for the coating solutions described below.

TABLE 1
Coating and cure conditions for forming (S2)

| | |
|---|---|
| Coating Width: | 6″ (15 cm) |
| Web Speed: | 30 feet (9.1 m) per minute |
| Solution % Solids: | 30.2% |
| Filter: | 2.5 micron absolute |
| Pressure Pot: | 1.5 gallon capacity (5.7 l) |
| Flow Rate: | 35 q/min |
| Wet Coating Thickness: | 24.9 microns |
| Dry Coating Thickness: | 4.9 microns |
| Conventional Oven Temps: | 140° F. (60° C.) Zone 1 |
| | 160° F. (53° C.) Zone 2 |
| | 180° F. (82° C.) Zone 3 |
| Length of Oven: | 10 feet (3 m) |

9. Preparation of High Index Optical Layer (S3):

ZrO₂ sol (Buhler Z-WO) (100.24 g 18.01% ZrO₂) was charged to a 16 oz jar. Methoxypropanol (101 g), Acryloxypropyl trimethoxy silane (3.65 g) and A1230 (2.47 g) were charged to a 500 ml beaker with stirring. The methoxypropanol mixture was then charged to the ZrO₂ sol with stirring. The jar was sealed and heated to 90 C for 4 hr. After heating the mixture was stripped to 52 g via rotary evaporation.

Deionized water (175 g) and concentrated NH₃ (3.4 g, 29 wt %) were charged to a 500-milliliter beaker. The above concentrated sol was added to this with minimal stirring. A white precipitate was obtained and isolated as a damp filter cake via vacuum filtration. The damp solids (43 g) were dispersed in acetone (57 g). The mixture was then filtered with fluted filter paper follow by 1-micron filter. The composition of the formed high index formulation, described in Table 2, was isolated at 15.8% solids.

TABLE 2

| wt % | Surface | | | | Wt % P.I. | |
|---|---|---|---|---|---|---|
| ZrO₂ | Modifier | wt % | wt % | Resins and | on total | % solids |
| nano | (SM) | SM | Resin | Ratios | solids | and solvent |
| 50% | 3:1 | 8.83 | 40.17 | Dipentaerythritol | 1.0% | 5% in |
| Buhler | Acrylate:A1230 | | | pentaacrylate | Irgacure ™ | acetone |
| | | | | (SR399) | 819 | |

The formulation was prepared at the % solids, in the solvent, and with the resins and photoinitiator indicated in the table above, by addition of the surface modified nanoparticles into a jar, followed by the addition of the available resins, initiator and solvents, followed by swirling to yield an even dispersion. (S3) was coated on the substrate (S2) using the same method and coating procedure but with the following parameters:

TABLE 3
Coating and cure conditions for forming (S3)

| | |
|---|---|
| Coating Width: | 4″ (10 cm) |
| Web Speed: | 10 feet per minute |
| Pump: | 60 cc Syringe Pump |
| Approximate Flow Rate: | 1.60 cc/min |
| Dry Coating Thickness: | 85 nm |
| UV cure Bulb | D-Bulb |
| Conventional Oven Temps: | 65° C. Zone 1 |
| | 65° C. Zone 2 |
| Length of Oven: | 10 feet (3 m) |

10. Preparation of High Index Optical Layer Substrate (S4):
   a. Nanoparticle preparation: (Buhler ZrO₂-75/25 acryloxypropyltrimethoxy silane-A1230)

The ZrO₂ sol (Buhler Z-WOS) (400.7 g of 23.03% ZrO₂) was charged to a 1 qt jar. Methoxypropanol (400 g), Acryloxypropyl trimethoxy silane (18.82 g) and A1230 (12.66 g) were charged to a 1-liter beaker with stirring. The methoxypropanol mixture was then charged to the ZrO₂ sol with stirring. The jar was sealed and heated to 90 degrees Celsius for 5.5 hours. After heating the mixture (759 g) was stripped to 230.7 g via rotary evaporation.

Deionized water (700 g) and concentrated NH₃ (17.15 g, 29 wt %) were charged to a 4 liter beaker. The above concentrated sol was added to this with minimal stirring. A white precipitate was obtained and isolated as a damp filter cake via vacuum filtration. The damp solids (215 g) were dispersed in methoxypropanol (853 g). The mixture was then concentrated (226 g) via rotary evaporation. Methoxypropanol (200 g) was added and the mixture concentrated (188.78 g ) via rotary evaporation. Methoxypropanol was charged (195 g) and the mixture was concentrated (251.2 g) via rotary evaporation. Methoxypropanol (130 g) was charged and the mixture concentrated via rotary evaporation. The final product (244.28) was isolated at 39.9% solids. The mixture was filtered thru a 1-micron filter. The high index coating solution has a composition as listed in Table 4:

TABLE 4

| wt % | Surface | | | Resins | Wt % P.I. | % solids |
|---|---|---|---|---|---|---|
| ZRO2 | Modifier | wt % | wt % | and | on total | and |
| nano | (SM) | SM | Resins | Ratios | solids | Solvent |
| 50 | 3:1 | 9 | 40 | 48:35:17 | 1.0% | 7.5% in |
| Buhler | Acrylate:A1230 | | | SR295:CN120Z:SR339 | Irgacure ™ | 10:1 |
| | | | | | 819 | Acetone:Methoxy |
| | | | | | | Propanol |

The formulation was prepared at the % solids, in the solvent, and with the resins and photoinitiator indicated in the table above, by addition of the surface modified nanoparticles into a jar, followed by the addition of the available resins, initiator and solvents, followed by swirling to yield an even dispersion. The high index coating solution was coated on the substrate (S2) using the same method and coating procedure described above but with the following parameters:

TABLE 5
Coating Conditions for the preparation of (S4):

| | |
|---|---|
| Coating Width: | 4″ (10 cm) |
| Web Speed: | 10 feet per minute |
| Pump: | 60 cc Syringe Pump |
| Approximate Flow Rate: | 1.18 cc/min |
| Dry Coating Thickness: | 85 nm |
| UV cure Bulb | D-Bulb |
| Conventional Oven Temps: | 65° C. Zone 1 |
| | 65° C. Zone 2 |
| Length of Oven: | 10 feet (3 m) |

C. Experimentation and Verification

The following paragraphs illustrate, via a specific set of example reactions and experimental methodologies, the improvements of each of the component steps for forming the low refractive index composition of the present invention.

### EXAMPLE 1

### Photocrosslinking/Photografting of Fluoropolymers

Fluoroplastic THV 220, Fluoroelastomer 2145 or Brominated Fluoroelastomer E-15742 were each dissolved individually in containers with either MEK or ethyl acetate at 10 weight percent by shaking at room temperature. The prepared fluoropolymer solutions were combined with one or more A174 or vinylsilane surface modified 20 nm sized silica particles as crosslinkers (Table 7) or alkoxysilyl substituted C═C double containing compounds/photografters (Table 8), in the presence of a photo-initiator, and without the presence of the amino-substituted organosilane ester or ester equivalent. The various compositions of coating solutions were allowed to sit in an airtight container. The solutions were then applied as a wet film to a PET or 906 hardcoated PET substrate. The coated films were dried in an oven at 100-120 degrees Celsius for 10 minutes.

Subsequently the films were subjected to UV (H-bulb) irradiation by 3 passes at the speed of 35 feet per minute. Alternatively, the films were subjected to UV irradiation from a 254 nanometer (nm) bulb using a similar approach. The resulting films were carefully removed from the coating substrates and cut into smaller pieces and placed into vials containing MEK solvent. The vials were visually observed to determine whether the film was soluble or insoluble in the MEK solvent. Solutions classified as “insoluble” indicated that the fluoropolymer was crosslinked, while solutions classified as “soluble” indicate that the solutions did not crosslink.

The following paragraphs describe the formation of the various evaluated materials contained in Tables 7 and 8.

Photochemical Reaction of Fluoropolymers with Vinylsilane or A174.

Brominated Fluoroelastomer E-15742, iodinated fluoroelastomer or THV200 were each dissolved individually in containers with either MEK or ethyl acetate at 10 weight percent by shaking at room temperature. The prepared fluoropolymer solutions were then combined with vinyl trimethoxysilane (or A174) in various ratios. The mixed fluoropolymer/Vinyl silane or fluoropolymer/A174 solutions were subsequently coated at a dry thickness of about 1-mil using a 20-mil thickness blocked coater onto PET, hardcoated PET, or polyimide film. The coated films were dried briefly, then subjected to heating at 100-140 degrees Celsius for 2 minutes.

Subsequently the films were subjected to UV (H-bulb) irradiation by 3 passes at the speed of 35 feet per minute. Alternatively, the films were subjected to UV irradiation from a 254-nm bulb using a similar approach. After UV irradiation, the cured films were removed from substrates and subsequently immersed into MEK solvent for dissolving the cured films. After overnight, the cured films were crosslinked by the residual amount of water from MEK or air and the films remained insoluble as indicated in Table 8 below.

Preparation of the Reaction Adduct of 1:1 Ratio of TMPTA and 3-aminopropyl triethoxysilane:

Into a flask having a magnetic stirrer was placed 29.6 g of TMPTA (0.1 mol). 221 g of 3-aminopropy triethoxysilane were slowly added to the TMPTA and reacted. The reaction gave off heat during the addition of the aminosilane. After stirring, the solution was allowed to sit for a few hours. Heating may be need to drive the reaction to completion. The reaction product was then diluted to a 10 weight percent solution with MEK.

Photochemical Reaction Products of Fluoropolymers with Alkoxysilyl Substituted Multiacrylates

Brominated Fluoroelastomer E-15742, iodinated fluoroelastomer or THV200 were each dissolved individually in containers with either MEK or ethyl acetate at 10 weight percent by shaking at room temperature. The prepared fluoropolymer solutions were then combined with the above prepared adduct of 1:1 molar ratio of TMPTA and 3-aminopropyl triethoxysilane. The mixed fluoropolymer/silyl substituted multiacrylate solutions were subsequently coated at a dry thickness of about 1-mil using a 20-mil thickness blocked coater onto PET, hardcoated PET or polyimide film. The coated films were dried briefly, then subjected to heating at 100-140 degrees Celsius for 2 minutes.

Subsequently the films were subjected to UV (H-bulb) irradiation by 3 passes at the speed of 35 feet per minute. Alternatively, the films were subjected to UV irradiation from a 254 nm bulb using a similar approach. After UV irradiation, the cured films were removed from substrates and subsequently immersed into MEK solvent for dissolving the cured films. After overnight, the cured films were crosslinked by the residual amount of water from MEK or air and the films remained insoluble as indicated in Table 8.

Photochemical Reaction Products of Fluoropolymers with Alkoxysilyl Substituted Multiacrylates in the Presence of Surface Functionalized Silica Particles

Brominated Fluoroelastomer E-15742, Iodinated fluoroelastomer or THV200 were each dissolved individually in containers with either MEK or ethyl acetate at 10 weight percent by shaking at room temperature. The prepared fluoropolymer solutions were then combined with the above-prepared adduct of 1:1 molar ratio of TMPTA and 3-aminopropyl triethoxysilane and A174/HMDS surface-modified 20 nm sized silica. The mixed fluoropolymer/silyl substituted multiacrylate/modified silica particle solutions were subsequently coated at a dry thickness of about 100 nm using a number 3 Meyer rod onto PET, a hardcoated PET or polyimide film. The coated films were dried briefly, then subjected to heating at 100-140 degrees Celsius for 2 minutes.

Subsequently the films were subjected to UV (H-bulb) irradiation by 3 passes at the speed of 35 feet per minute. The cured films were evaluated by rubbing with kimwipe for 10 times.

Tables 6 and 7 confirmed that the fluoropolymers reacted with either the listed crosslinkers or grafting agents, as confirmed by the visual observation of insolubility of the liquid in the vials.

TABLE 6
Photocrosslinking/photografting of fluoropolymers aided
by functionalized particles and photo-initiators

| | Photo- | | | |
|---|---|---|---|---|
| Fluoropolymer | initiator | Crosslinker | UV | Observations |
| E15742 | KB-1 | — | 254 nm | Slightly insoluble |
| E15742 | KB-1 | F—SiO₂ | 254 nm | Insoluble |
| | | (vinyl/HMDS) | | |
| E15742 | 1173 | F—SiO₂ | 254 nm | Insoluble |
| | | (vinyl/HMDS) | | |
| E15742 | 1173 | F—SiO₂ | 254 nm | Insoluble |
| | | (A174/HMDS) | | |
| E15742 | KB-1 | F—SiO₂ | H- | Insoluble |
| | | (vinyl/HMDS) | bulb | |
| E15742 | 1173 | F—SiO₂ | H- | Insoluble |
| | | (vinyl/HMDS) | bulb | |
| E15742 | 1173 | F—SiO₂ | H- | Insoluble |
| | | (A174/HMDS) | bulb | |

TABLE 7
Photografting of Vinyl silane or A174 onto
fluoropolymers aided by photo-initiators

| | Photo- | Grafting | | |
|---|---|---|---|---|
| Fluoropolymer | initiator | agent | UV | Observations |
| E15742 (98) | KB-1 | Vinyl silane | H- | Insoluble |
| | | (2) | bulb | |
| E15742 (98) | 1173 | Vinyl silane | H- | Insoluble |
| | | (2) | bulb | |
| E15742 (95) | KB-1 | Vinyl silane | H- | Insoluble |
| | | (5) | bulb | |
| E15742 (95) | 1173 | Vinyl silane | H- | Insoluble |
| | | (5) | bulb | |
| E15742 (98) | KB-1 | A174 (2) | H- | Insoluble |
| | | | bulb | |
| E15742 (98) | 1173 | A174 (2) | H- | Insoluble |
| | | | bulb | |
| E15742 (95) | KB-1 | A174 (5) | H- | Insoluble |
| | | | bulb | |
| E15742 (95) | 1173 | A174 (5) | H- | Insoluble |
| | | | bulb | |

### EXAMPLE 2

### Scratch Resistance Improved by Grafting Agents, Bonding Promoters, Alkoxysilyl Substituted Mono- or Multi-Functional Crosslinkers and Inorganic Nanoparticles

The above prepared fluoropolymer solutions were also combined with inorganic nanoparticles which had been surface modified by either 3-(trimethoxysilyl)propyl methacrylate or vinyltrimethoxysilane in various ratios. The fluoropolymer/nanoparticle solutions were further combined with TMPTA, MMA, aminosilane and a photo-initiator in various ratios. The various compositions of coating solutions (Table 8) were allowed to diluted to either a 3 or 5 weight percent solution and allowed to sit in a container. The reaction product was then coated at a dry thickness of about 100 nm using a number 3 wire wound rod as a wet film to a PET or hardcoated PET substrate. The coated films were dried in an oven at 100-140 degrees Celsius for 2 minutes.

Subsequently the films were subjected to UV (H-bulb) irradiation by 3 passes at the speed of 35 feet per minute. Alternatively, the films were subjected to UV irradiation from a 254 nm bulb using a similar approach. The scratch resistance of the film samples, which is an indicator of good interfacial adhesion between the film and the substrate, was tested by rubbing with paper towel.

As shown in Table 8, the resulting films showed excellent interfacial adhesion, especially in samples utilizing the aminosilane or A1106 adhesion promoter to the PET substrate or hardcoated PET substrate. Further, irradiation of the various samples resulted in improved interfacial adhesion in Table 8.

Improved Scratching Resistance by Photocrosslinking or by Photografting

The above prepared fluoropolymer solutions were combined with TMPTA, MMA, HFPO mono or multiacrylates or combinations, aminosilane and a photo-initiator in various ratios. The various compositions of coating solutions (Table 8) were allowed to diluted to either 3 or 5 weight percent solutions and allowed to sit in a container. The reaction products were then coated at a dry thickness of about 100 nm using a number 3 wire wound rod as a wet film to a PET or hardcoated PET substrate. The coated films were dried in an oven at 100-140 degrees Celsius for 2 minutes.

Subsequently the films were subjected to UV (H-bulb) irradiation by 3 passes at the speed of 35 feet per minute. Alternatively, the films were subjected to UV irradiation from a 254 nm bulb using a similar approach. The scratch resistance of the film samples, which is an indicator of good interfacial adhesion between the film and the substrate, was tested by rubbing with a paper towel.

As shown in Table 8, the resulting films showed excellent interfacial adhesion and scratching resistance, especially in fluoropolymer samples utilizing the aminosilane or A1106 adhesion promoter to the PET substrate or hardcoated PET substrate. Further, irradiation of the various samples resulted in improved interfacial adhesion.

TABLE 8
Improvement of scratch resistance of fluoropolymer films
by adhesion promoters, photocrosslinkers, photografting
agents and functionalized inorganic nanoparticles

| Fluoropolymer/ | | | | |
|---|---|---|---|---|
| Adhesion | Crosslinker/ | Photo- | UV | |
| Promoter | Grafting agent/ | Initiator | (35 feet/min 3 | |
| (95:5; W %) | Monomer | (1 wt %) | passes) | Observations |
| THV220 | | | H-bulb | Film peeling off |
| THV220/A1106 | | | H-bulb | Some scratching |
| THV220/A1106 | VS (5%) | 1173 | H-bulb | No scratching |
| (95) | | | | |
| THV220/A1106 | VS (2%) | 1173 | H-bulb | No scratching |
| (98) | | | | |
| THV220/A1106 | A174 (5%) | 1173 | H-bulb | Slight scratching |
| (95) | | | | |
| THV220/A1106 | VS (5%) | KB-1 | H-bulb | Scratching |
| (95) | | | | |
| THV220/A1106 | A174 (5%) | KB-1 | H-bulb | Scratching |
| (95) | | | | |
| E15742 | | | H-bulb | Film peeling off |
| E15742/A1106 | | | H-bulb | Some scratching |
| E15742/A1106 | VS(5) | 1173 | H-bulb | No scratching |
| (95) | | | | |
| E15742/A1106 | VS(8) | KB-1 | H-bulb | No scratching |
| (92) | | | | |
| E15742/A1106 | VS(10)/TMPTA | 1173 | H-bulb | No scratching |
| (95) | (10) | | | |
| E15742/A1106 | TMPTA (1)/A174 | 1173 | H-bulb | Slight scratching |
| (97) | (2) | | | |
| E15742/A1106 | TMPTA (5)/A174 | KB-1 | H-bulb | No scratching |
| (97) | (5) | | | |
| E15742/A1106 | TMPTA (5)/A174 | KB-1 | No | Scratching |
| (97) | (5) | | | |
| E15742/A1106 | A174 (5) | 1173 | H-bulb | No scratching |
| (95) | | | | |
| E15742/A1106 | A174 (8) | KB-1 | H-bulb | No scratching |
| (92) | | | | |
| E15742/A1106 | VS (3)/TEOS(15) | 1173 | H-bulb | Slight scratching |
| (82) | | | | |
| E15742/A1106 | A174-SiO2 (10) | 1173 | H-bulb | Slight scratching |
| (90) | | | | |
| E15742/A1106 | VS-SiO2 (10) | 1173 | H-bulb | Slight scratching |
| (90) | | | | |
| E158/A1106 (95) | VS(5) | KB-1 | H-bulb | No scratching |
| E158/A1106 (95) | A-174 (5) | KB-1 | H-bulb | No scratching |
| E15742(90) | TMPTA:3-APS = 1:1 | KB-1 | H-bulb | Some scratching |
| | adduct(10) | | | |
| E15742(60) | TMPTA:3-APS = 1:1 | KB-1 | H-bulb | Some scratching |
| | adduct(10), A174- | | | |
| | SiO2(30) | | | |
| E18402(90) | TMPTA:3-APS = 1:1 | KB-1 | H-bulb | Some scratching |
| | adduct(10) | | | |
| E18402(60) | TMPTA:3-APS = 1:1 | KB-1 | H-bulb | Some scratching |
| | adduct(10), A174- | | | |
| | SiO2(30) | | | |
| THV220(90) | TMPTA:3-APS = 1:1 | KB-1 | H-bulb | Some scratching |
| | adduct(10) | | | |
| THV220(60) | TMPTA:3-APS = 1:1 | KB-1 | H-bulb | Some scratching |
| | adduct(10), A174- | | | |
| | SiO2(30) | | | |
| E15742(60) | TMPTA:A1106 = 1:1 | KB-1 | H-bulb | Some scratching |
| | adduct(10), A174- | | | |
| | SiO2(30) | | | |
| E15742(50) | TMPTA:A1106 = 1:1 | KB-1 | H-bulb | Some scratching |
| | adduct(10), | | | |
| | TMPTA(10), A174- | | | |
| | SiO2(30) | | | |

A1106 = oligomers of 3-aminopropyltriethoxylsilane
VS = Vinyl trimethoxylsilane
A174 = 3-(trimethoxysilyl)propyl methacrylate
E15742 = bromine-containing fluoroelastomer
E18402 = iodine-containing fluoroelastomer

### EXAMPLE 3

### Refractive Index Measurements of Samples Showing Improved Scratch Resistance in Tables IV and V

For samples in Table 9 that showed improved scratch resistance, refractive index measurements were performed to confirm the resultant coatings usefulness as a low refractive index layer, wherein the measure refractive index is below 1.4.

As Table 9 indicates, each of the scratch resistant samples tested measured less than 1.4, and thus were suitable for use in a low refractive index layer of an antireflection film.

TABLE 9
Refractive indices of such fluoropolymer films with
improved scratch resistance

| Fluoropolymer/ | Crosslinker/ | | | | |
|---|---|---|---|---|---|
| Adhesion | Grafting | Photo- | Wave- | | |
| Promoter | Agent/ | Initiator | length | Refractive | |
| (95:5; W %) | Monomer | (1 w %) | (nm) | Indice | K |
| E15742/A1106 | Vinylsilane(5) | 1173 | 533.567 | 1.3457 | 0.01844 |
| (95) | | | | | |
| E15742/A1106 | A174(15)/TMP | 1173 | 533.567 | 1.3556 | 0.02109 |
| (80) | TA(5) | | | | |
| E15742/A1106 | A174(5) | 1173 | 533.567 | 1.3740 | 0.00856 |
| (95) | | | | | |
| E15742/A1106 | Vinylsilane(10) | 1173 | 533.567 | 1.3777 | 0.0094 |
| (90) | | | | | |

Next, in Table 10, various coatings were applied at a dry thickness of about 100 nm using a number 3 wire wound rod as a wet film to a to a zirconium high index coated substrate. A 10 weight percent coating concentration was applied to the substrate to a 10-mil thickness. The film was heated at 140 degrees Celsius for 1 minute. The heated film was then subjected to 3 passes under a UV lamp for samples with E15742 and 2 passes samples with E18402 and THV220. A peel test measurement, which is an indicator of the amount of interfacial adhesion between the coated film and the substrate, was performed on each sample by the test method described above previously. As the testing indicated, the resulting films having aminosilane and A1106 adhesion promoter had improved interfacial adhesion to the zirconium substrate.

TABLE 10
Peel Strength Measurement Table V (lbs/in): Fluoropolymer
coating adhesion to ZrO₂ high index coated substrate

| | Average of | Average of |
|---|---|---|
| Coating Sample | Average | Maximum |
| E15742 | 0.3 | 0.4 |
| E15742 + A-174 (95:5) | 2.0 | 2.4 |
| E15742 + Vinylsilane (95:5) | 1.5 | 1.9 |
| E15742 + A-174 + A1106 (90:5:5) | 2.0 | 2.4 torn |
| | | sample |
| E15742 + A-174 + 3-APS (90:5:5) | 1.4 | 1.7 |
| E15742 + Vinylsilane + A1106 (90:5:5) | 1.7 | 2.1 |
| E15742 + Vinylsilane + 3-APS (90:5:5) | 2.0 | 3.4 |
| E15742 + A-174 (90:10) | 2.2 | 2.8 |
| E15742 + Vinylsilane (90:10) | 1.3 | 1.6 |
| E18402 | 0.9 | 1.1 |
| E18402 + A-174 (95:5) | 2.8 | 4.4 |
| THV220 | 0.6 | 1.0 |
| THV220 + Vinylsilane (95:5) | 2.8 | 4.0 |

While the invention has been described in terms of preferred embodiments, it will be understood, of course, that the invention is not limited thereto since modifications may be made by those skilled in the art, particularly in light of the foregoing teachings.

What is claimed is:

1. A low refractive index composition for use in an antireflection coating for an optical display, the composition comprising the reaction product of:
a fluoropolymer;
a C═C double bond containing silane ester agent;
a plurality of surface modified nanoparticles; and
optionally a multi-olefinic crosslinker.

2. The composition of claim 1, wherein said multi-olefinic crosslinker comprises a multi-acrylate crosslinker.

3. The composition of claim 1, wherein said fluoropolymer is selected from the group consisting of a VDF containing homopolymer, a VDF copolymer, a TFE copolymer, a HFP copolymer, THV, and FKM.

4. The composition of claim 1, wherein said fluoropolymer comprises a fluoroelastomer.

5. The composition of claim 4, said fluoroelastomer is selected from the group consisting of a Cl-containing fluoroelastomer, Br-containing fluoroelastomer, an I-containing fluoroelastomer, and a CN-containing fluoroelastomer.

6. The composition of claim 2, wherein said multi-acrylate crosslinker comprises a fluorinated multi-acrylate crosslinker.

7. The composition of claim 6, wherein said fluorinated multi-acrylate crosslinker comprises a perfluoropolyether multi-acrylate crosslinker.

8. The composition of claim 7, wherein said perfluoropolyether multi-acrylate crosslinker comprises an HFPO-multiacrylate crosslinker.

9. The composition of claim 2, wherein said multi-acrylate crosslinker is selected from the group consisting of PETA and TMPTA.

10. The composition of claim 2, wherein said multi-olefinic crosslinker further comprises a mono-acrylate.

11. The composition of claim 10, wherein said mono-acrylate comprises a fluorinated mono-acrylate.

12. The composition of claim 11, wherein said fluorinated mono-acrylate comprises a perfluoropolyether mono-acrylate.

13. The composition of claim 12, wherein said perfluoropolyether mono-acrylate comprises an RFPO-monoacrylate.

14. The composition of claim 1, wherein said a C═C double bond containing silane ester agent comprises a vinyl silane ester compound.

15. The composition of claim 1, wherein said C═C double bond containing silane ester compound comprises 3-(trimethoxysilyl) propyl methacrylate.

16. The composition of claim 14, wherein said vinyl silane ester compound comprises vinyltrimethoxy silane.

17. The composition of claim 14, wherein said C═C double bond containing silane ester agent are polymeric oligomers.

18. The composition of claim 1, wherein said multi-olefinic crosslinker comprises an alkoxysilyl-containing multi-olefinic crosslinker

19. An antireflection film having a layer of said low refractive index material of claim 1, said antireflection film further comprising a high refractive index layer coupled to said layer of said low refractive index material.

20. An optical device comprising a layer of said low refractive index material formed according to claim 1.

21. A low refractive index composition for use in an antireflection coating for an optical display, the composition comprising the reaction product of:
a fluoropolymer; and
an alkoxysilyl-containing multi-olefinic crosslinker.

22. The composition of claim 21 further comprising a plurality of surface modified inorganic particles.

23. An antireflection film having a layer of said low refractive index material of claim 21, said antireflection film further comprising a high refractive index layer coupled to said layer of said low refractive index material.

24. An optical device comprising a layer of said low refractive index material formed according to claim 21.

25. A method for forming an optically transmissive, stain and ink repellent, durable optical display comprising:
providing an optical display having an optical substrate;
forming a low refractive index polymer composition comprising a fluoropolymer, a C═C double bond containing silane ester agent, and an alkoxysilyl-containing multi-olefinic crosslinker;
applying a layer of said low refractive index polymer composition to said optical substrate; and
curing said layer to form a cured film.

26. The method of claim 25, wherein providing an optical display comprises providing an optical display having a hard coat layer applied to an optical substrate

27. The method of claim 25, wherein forming a low refractive index polymer composition comprises:
reactively coupling a fluoropolymer and a C═C double bond containing silane ester agent to form an silyl functional fluoropolymer; and
introducing a alkoxysilyl-containing multi-olefinic crosslinker to said silyl functional fluoropolymer.
